(19) United States
(12) Patent Application Publication
(10) Pub. No.: US 2005/0147989 A1
(43) Pub. Date: Jul. 7, 2005

(54) Screening assay for aggregations

(76) Inventors: Uwe Bertsch, Scheuring (DE); Armin Giese, Muenchen (DE); Hans A. Kretzschmar, Wolfratshausen (DE); Jan Bieschke, San Diego, CA (US)

Correspondence Address: JACOBSON HOLMAN PLLC, 400 SEVENTH STREET N.W., SUITE 600, WASHINGTON, DC, 20004, US

(21) Appl. No.: 10/954,361

(22) Filed: Oct. 1, 2004

(30) Foreign Application Priority Data
Oct. 2, 2003 (EP) 03 022 096.6
Mar. 12, 2004 (EP) 04 005 940.4

(51) Int. Cl. C12Q 1/68; G01N 33/53; G01N 33/543

(52) U.S. Cl. 435/6; 435/7.1; 436/523

(57) ABSTRACT

A method for the identification of substances for influencing aggregations comprising the following steps: a) combining at least one aggregate having a first detectable function and at least one monomer having a second detectable function, wherein said at least one monomer has an affinity for said at least one aggregate, in the presence of a potentially aggregation-influencing substance; b) determining a degree of labeling of the aggregates, the degree of labeling being a measure of the number and proportion of the detectable functions bound.

The present invention relates to a screening assay for the identification of substances for influencing aggregations.

A number of different diseases is associated with the occurrence of pathological depositions (aggregates), especially protein aggregates. Thus, neurodegenerative diseases are known in which, for example, protein depositions referred to as amyloid plaques can be detected in the brain of afflicted persons. Such diseases include, for example, Alzheimer's disease, bovine spongiform encephalopathy (BSE), Creutzfeldt-Jakob disease (CJD), laughing death syndrome, scrapie. Recently, the BSE disease, in particular, has become a focus of public attention, which is due to the fact, inter alia, that BSE has been connected with the Creutzfeldt-Jakob disease in humans. Today, the mechanisms by which the protein depositions affect the pathological process are still unclear. The relationship, observed by Prusiner, between infectiosity and the concentration of certain proteins which play a role in the pathological process of scrapie, a neurodegenerative disease in sheep, is remarkable. Pathological protein depositions appear not only in diseases of the neuronal system, but are observed in other organs as well, such as in a disease of diabetes type II.

A survey of prion diseases has been published by D. Riesner in “Chemie in unserer Zeit” (1996), p. 66-74. Especially for Alzheimer's disease, the pathological picture has been described relatively well. “Senile plaques”, which substantially consist of aggregated amyloid-β protein, and “paired helical filaments”, which are constituted of abnormally altered tau protein, are closely connected with Alzheimer's disease.

Therefore, the objective of a causal therapy should be to prevent or reduce aggregation.

The previous methods for searching for therapeutic agents for amyloid-caused neurodegenerative diseases are based on either tedious in vivo test methods in afflicted animals (or animal models produced by genetic engineering) or in (infected) cell cultures, or in vitro methods, which are also tedious and frequently accompanied by the radioactive labeling of proteins and long incubation phases (from 1 to 7 days). As a method for evaluating the effectiveness of substances, Western blot methods are frequently employed which consist of several electrophoretic and incubation steps, which often take hours, and usually only allow for a limited number of samples in one run (<30).

For in vivo experiments, comparatively large amounts of the substances to be examined are needed because effective concentrations of up to 10 μM must be maintained in an animal or in several milliliters of a cell culture dish often over several days or weeks.

The object of the invention is to provide a method for the identification of substances for influencing aggregations which overcomes the above mentioned drawbacks and, in particular, enables a high throughput for small amounts of substance and a substantially automatable method.

This object is achieved by a method with the features of claim 1.

The method according to the invention for the identification of substances for influencing aggregations comprises the following steps:
a) combining at least one aggregate having a first detectable function and at least one monomer having a second detectable function, wherein said at least one monomer has an affinity for said at least one aggregate, in the presence of a potentially aggregation-influencing substance;
b) determining a degree of labeling of the aggregates.

The degree of labeling is a measure of the number and proportion of the detectable functions bound.

According to the invention, an aggregate which has a detectable function is incubated together with a monomer having a second detectable function which can be distinguished from the first. Said monomer must be capable of binding to the aggregate. The incubation is effected in the presence of a potentially aggregation-influencing substance. The inhibition of the binding of the monomer to the aggregate is determined by measuring the aggregates with respect to both the first and the second detectable functions.

“Aggregates” within the meaning of this application refers to an aggregation of structures of essentially similar constituents and binding capabilities for further units. In one embodiment, these are pathological protein aggregates. Typical protein aggregates consist of the components of the prior protein, APP, tau, α-synuclein or proteins having a polyglutamine sequence, such as huntingtin, fragments or derivatives of such proteins.

Alternatively, the aggregates may also consist of other constituents, especially nucleic acids, lipids, polysaccharides, vesicular systems, or nanoelements.

The aggregates employed according to the invention are capable of binding the units referred to as “monomers”.

“Monomer” refers to a structure which is capable of binding to aggregates and may be a component of complexes of more than one monomeric constituent.

In a preferred embodiment, the monomer and the aggregate have similar chemical structures, i.e., the aggregate is constituted of components which are either identical with the monomer or have a similar biological function, but are, for example, a fragment, derivative etc., for example, having a slightly deviating chemical structure.

A “detectable function” within the meaning of this application is a function which can be specifically detected in the method. Typical detectable functions include, for example, radioactive labeling, dye labeling, such as fluorescence labeling. The detectable function may be bound directly to the aggregate or monomer, but it may also be bound indirectly (secondary labeling), for example, by binding aggregates or monomers to an antibody which in turn has itself a detectable function, such as a fluorescence molecule etc.

The first and second detectable functions can be distinguished according to the invention.

“Particles” within the meaning of this application are units which are detectable, especially the aggregates, monomers or complexes of aggregates with one or more monomers, optionally together with other molecules, for example, antibodies.

In a particularly preferred embodiment, the first detectable function is bound to the aggregate through a binding molecule, said binding molecule having a high affinity for aggregates and a low affinity for monomers. The terms “high” and “low” are to be understood in a relative way, i.e., the affinity for the aggregates must be higher than that for the monomers. The absolute degree of affinity is less important. Typically, the affinity for the aggregate (K_{D} value) is different from the affinity for the monomer by at least a factor of 10.

Preferred binding molecules are, for example, antibodies, fragments of antibodies or recombinant molecules having the binding function of an antibody, such as scFv fragments.

Particularly suitable detectable functions are fluorescent molecules. Only the first, only the second or both detectable functions may be in the form of fluorescent molecules.

According to the invention, it is particularly advantageous if the proportion of aggregates is determined on the basis of individual particles, i.e., the measurement ensures that a high number of aggregates can be measured individually each.

Advantageously, the number and proportion of all detectable functions may also be measured, especially of all detectable functions which are bound to particles. One particularly suitable measuring method for this purpose is the method described in detail in WO 01/23894. This application is included herein by reference. Details will be illustrated below.

In a particularly preferred embodiment, the method according to the invention is employed for the search for active substances for treating protein aggregation diseases. “Protein aggregation diseases” is understood to mean both diseases in which the aggregation is primary (e.g., prion diseases) and diseases in which the aggregation is secondary, but contributes to the tissue damage. The aggregate is a protein aggregate, and the monomer is a protein monomer, and the substance which influences the aggregation is a potential active substance for the treatment of a protein aggregation disease.

In this case, the protein aggregate may be a multimer of the protein monomer. However, they may also be structurally different as long as there is still affinity between the protein aggregate and the protein monomer.

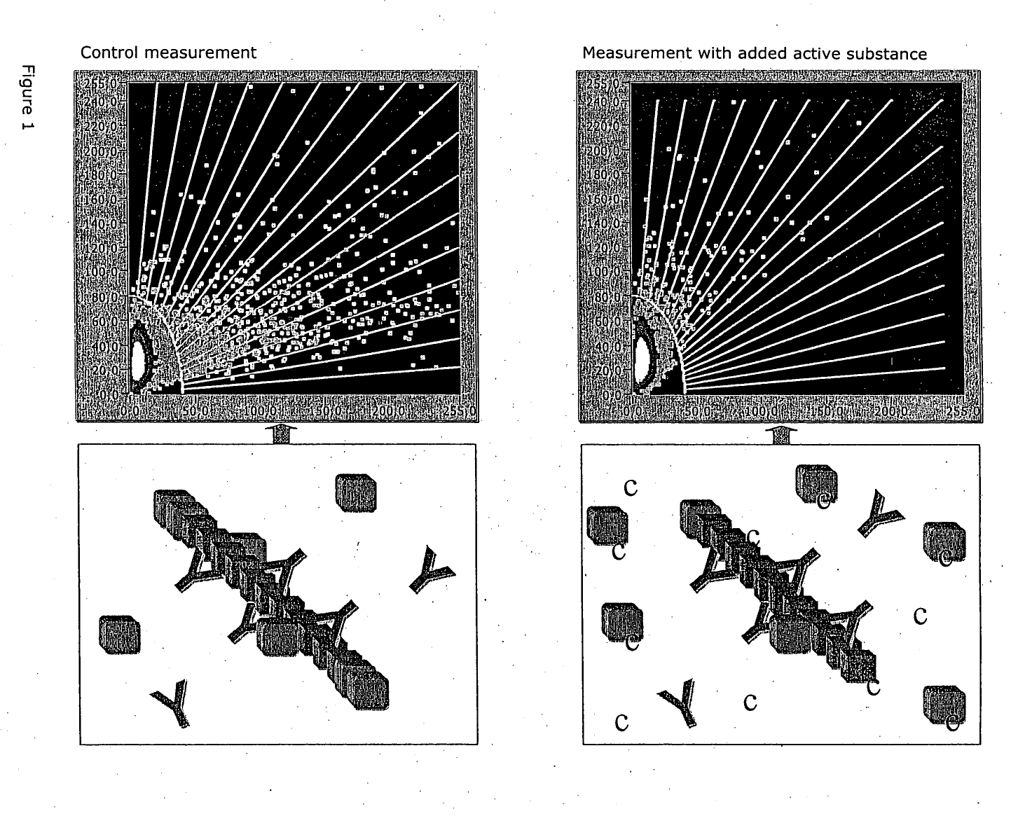

The method according to the invention is explained with reference to FIG. 1. In a control measurement, aggregates (shown as interconnected dark units in the Figure) are incubated together with monomers (light units). The light units carry a detectable function, for example, a fluorescence dye. The protein aggregates are labeled with a second dye by adding an antibody which is specific for the aggregates, but virtually does not bind to the monomers. Depending on the concentration ratios, a proportion of particles which carry only the first label or only the second label or particles which carry both labels in some ratio is established. For evaluation, for example, as shown in FIG. 1, the degrees of labeling of individual particles are plotted against each other on two axes.

In the right-hand graph, a potentially active substance “C” has been added. By binding to the monomer, it partially prevents binding to the aggregate. This results in a change of the labeling on the aggregates and concurrently in a corresponding shift in the fluorescence pattern.

The method according to the invention as described above is suitable for searching for active substances, but also as a general assay for the identification of substances which influence aggregation.

The method according to the invention is also suitable for analyzing any other aggregates, for example, of nucleic acids, vesicles or nanoelements. Nanoelements are organic or inorganic molecules which form larger structures in a self-assembling process. Nanoelements include, for example, fullerenes or molecule/atom clusters (prepared in a pure or mixed form).

In particular, aggregates may be measured in which the aggregates and monomers originate from different classes of substances, for example, nucleic acids+protein, as with ribosomes or histone-packed DNA, any vesicular micellary or supporting structures, or the binding of substances to polysaccharides.

In principle, the measuring of aggregation may be effected in cell-free systems, in a cell, or else in the supernatant of a cell culture.

Different techniques are suitable as measuring methods. A possibility are imaging methods, especially in the case of fluorescent or other dye-labeled particles, for example, an Opera® system from Evotec Technologies.

A particularly suitable method is described in WO 01/23894, referred to as SIFT (Scanning for Intensely Fluorescent Targets) in an advantageous embodiment. It is based on a time-dissolved intensity analysis of a fluorescent signal in an open volume element which is defined by a confocal figure of one or more excitation lasers bundled in one focus. This enables a quantification of the particle-derived signal fraction, preferably by analyzing the intensity distribution of a measured detection signal, for example, a fluorescence signal, in successive time windows.

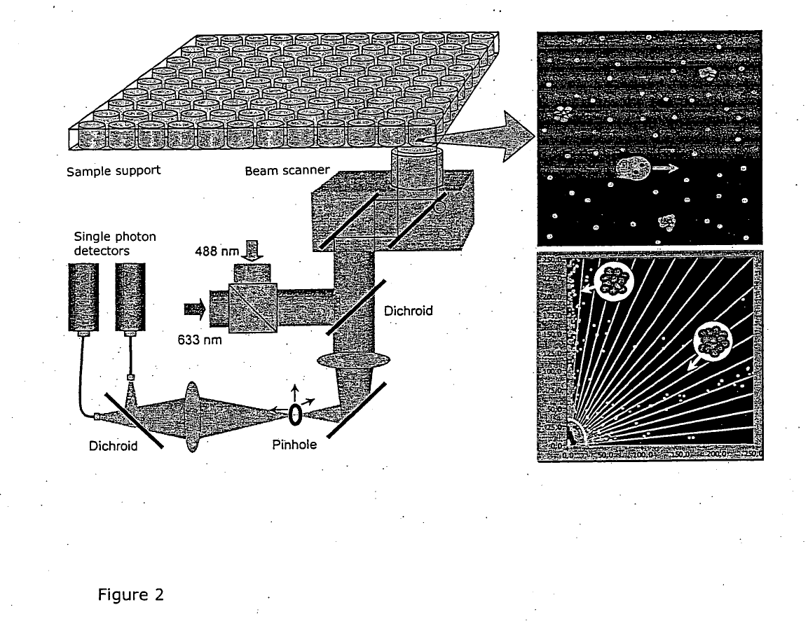

These time windows are referred to as “bins”. Typical detection times are in a microseconds to milliseconds range and may have constant or variable lengths, whereby the very intensive signal from the multiply labeled particles can be separated from the background signal. The scanning of the sample may be supported by an essentially constant relative movement between the sample and the measuring volume. This increases the volume examined and thus the measuring sensitivity. Especially for slowly diffusing particles, this has advantages because the dwelling time is no longer determined by the diffusion time, but by the scanning speed. A typical set-up is shown in FIG. 2.

This method has also been described in Bieschke et al., PNAS (2000), 5468 to 5473.

The method according to the invention is further illustrated by the following example.

### EXAMPLE 1

Assay for Anti-Prion Substance

Based on the SIFT technique and using a fluorescence correlation spectroscope, the association reaction between recombinantly prepared mouse PrP (amino acids 23-231) and PrP-Sc aggregates prepared from the brain tissue of CJD patients (here: according to the method by Safar et al., Nat. Med. 1998 October; 4 (10): 1157-65) is examined. For this association reaction, the recombinant mouse PrP is labeled with a fluorescent dye (here: Alexa488) covalently on lysine residues. In contrast, the PrP-Sc aggregates are labeled by adding a monoclonal antibody (here: L42 [Vorberg et al., Virology, 1999, March 1; 255 (1): 26-31]) directed against human PrP and labeled with a second fluorescent dye (here: Alexa647). The antibody employed will bind to human PrP-Sc, but not to the added recombinant mouse PrP.

The PrP-Sc aggregates which carry a large number of fluorophors due to the binding of many monoclonal antibodies and thus show a strong fluorescence also aggregate the recombinant mouse PrP in large numbers under the defined conditions (here: 20 mM potassium phosphate buffer, pH 6.0; 0.1% Nonidet-P40). This produces aggregates of human PrP-Sc and fluorescence-labeled antibody as well as fluorescence-labeled mouse PrP, which therefore exhibit fluorescence intensities for both dyes. Such aggregates showing highly intense two-colored fluorescence can be detected quantitatively with the SIFT technique in the FCS device employed, and distinguished from any occurring aggregates of the antibodies or the mouse PrP alone. In the two-dimensional SIFT evaluation, which is preferably used according to the invention, the measuring times observed are plotted against each other in a two-dimensional intensity histogram in accordance with the respectively measured fluorescence intensity of the two dyes, and from this, a measure of the quantity of aggregates having particular labeling conditions of the two detected probe species is established. The quantification is typically done in an automated manner by summing up all measuring times above a threshold value with similar color intensity ratios, which are summed up by sectors in accordance with their attribution to an angular range of the two-dimensional SIFT diagram.

Now, in this screening method for anti-prior drugs, substances are added to this association reaction between PrP-Sc and mouse PrP (a typical final concentration used herein is around 10 μM), and the prevention of aggregation of the mouse PrP to the PrP-Sc aggregates is analyzed. Substances which prevent this aggregation cause a shifting of the ratio of the fluorescence intensities of the two dyes in the two-dimensional SIFT evaluation for the detectable aggregates towards the color of the antibody. In the extreme case of a complete suppression of the mouse PrP binding to the aggregates, the latter only shine in the color of the antibody.

The method according to the invention is substantially automatable and allows to detect the effectiveness of a substance with a short measuring time (typically less than 75 s per substance) and a quantitative evaluation of the result which is performed directly on line. Another particular advantage of the method resides in the fact that it works with extremely small quantities of substance. Thus, measuring a substance in a volume of 20 μl at a final concentration of 10 μM requires only 0.2 nmol of the substance.

As preparatory operations for the method, typically, only the purification of protein aggregates and the fluorescence labeling of antibodies and bacterially prepared and purified protein monomer cannot be automated completely. However, these preliminary operations are performed for a large number of individual measurements in one run, so that the working time required per individual measurement is hardly significant. The actual screening assay can then be measured directly as a homogeneous assay according to the “mix and measure” principle without further separating steps.

### EXAMPLE 2

Validation of the Method

The method was validated with a model substance known to influence the formation of PrP-Sc during the infectious process in vivo.

As a potential prior therapeutic agent, the polycationic lipid 2,3-dioleyloxy-N-[2-(sperminecarboxamido)ethyl]-N,N-dimethyl-1-propanaminium trifluoroacetate (DOSPA) was used. It was found that it inhibits the association of fluorescence-labeled recombinant PrP to prion aggregates at low micromolar concentrations. In controls with other lipids, such as dioleyl-L-phosphatidylethanolamine (DOPE), no influence on aggregation was found, as expected.

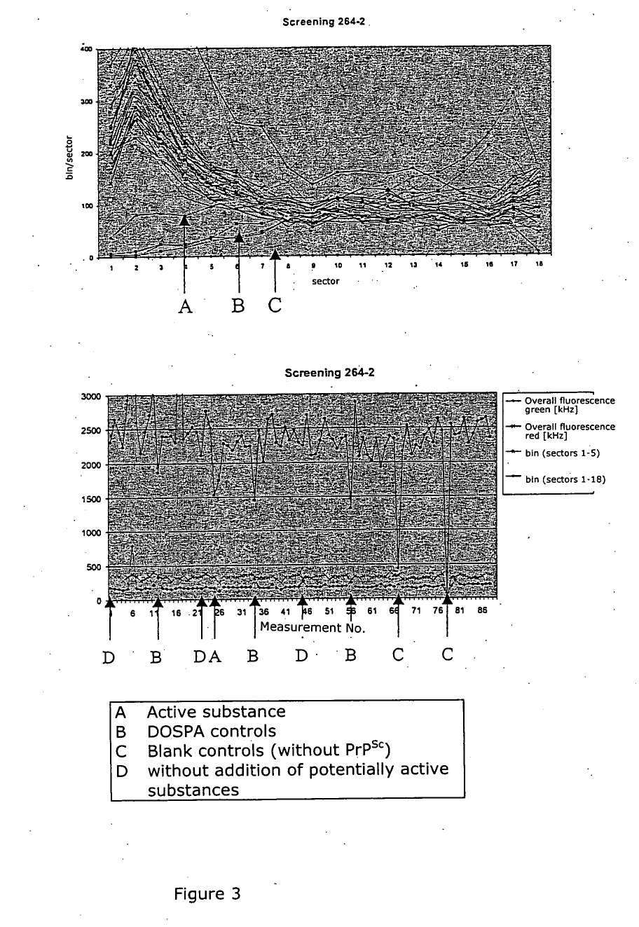

FIG. 3 shows a screening assay for 80 substances and eight controls in a microtitration plate format.

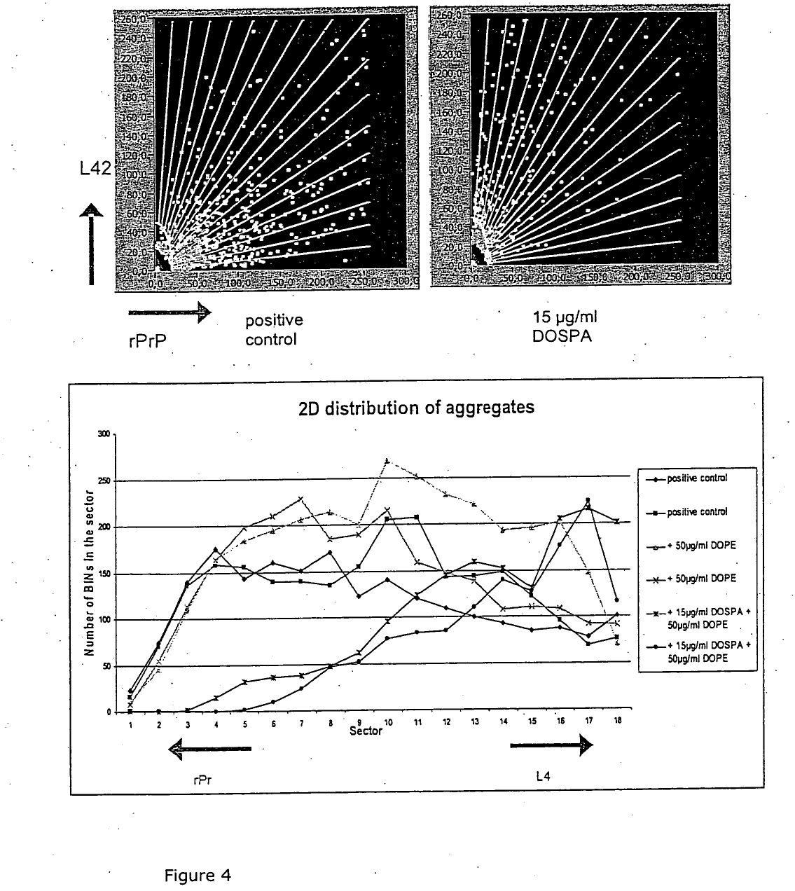

FIG. 4 shows the results of the examinations with DOSPA. Thus, the two-dimensional histograms of the fluorescence intensity are analyzed quantitatively by counting the number of bins in each sector. In the evaluation, it becomes clear that the number of bins is decreased in sectors 1 to 9.

1. A method for the identification of substances for influencing aggregations comprising the following steps:
a) combining at least one aggregate having a first detectable function and at least one monomer having a second detectable function, wherein said at least one monomer has an affinity for said at least one aggregate, in the presence of a potentially aggregation-influencing substance;
b) determining a degree of labeling of the aggregates, the degree of labeling being a measure of the number and proportion of the detectable functions bound.

2. The method according to claim 1, characterized in that said first detectable function is bound to a binding molecule, said binding molecule having a high affinity for said at least one aggregate and a low affinity for said at least one monomer.

3. The method according to claim 2, characterized in that said binding molecule is an antibody, a fragment of an antibody or a recombinant molecule having the binding function of an antibody.

4. The method according to claim 1, characterized in that said first and/or second detectable function is a fluorescence molecule.

5. The method according to claim 1, characterized in that the degree of labeling of aggregates is determined on the basis of individual particles.

6. The method according to claim 1, characterized in that the numbers and proportions of all detectable functions are measured.

7. The method according to claim 1, characterized in that said measurement is effected by means of the SIFT technique.

8. The method according to claim 1, characterized in that said aggregate is selected from the group consisting of proteins, nucleic acids, lipids, polysaccharides, vesicular systems and nanoelements.

9. The method according to claim 1, characterized in that said monomer is selected from the group consisting of proteins, nucleic acids, lipids, polysaccharides, vesicular systems and nanoelements.

10. The method according to claim 1, characterized in that said aggregate is a multimer of said monomer.

11. A kit containing at least one aggregate having a first detectable function and at least one monomer having a second detectable function.
